(19) United States
(12) Patent Application Publication
(10) Pub. No.: US 2022/0165424 A1
(43) Pub. Date: May 26, 2022

(54) METHOD AND SYSTEM FOR MANAGING A PARTICIPANT HEALTH REGIMEN

(71) Applicant: Omada Health, Inc., San Francisco, CA (US)

(72) Inventors: Carolyn Jasik, San Francisco, CA (US); Mike Tadlock, San Francisco, CA (US); Michael Turken, San Francisco, CA (US); Ken Huang, San Francisco, CA (US); Jimmy Xu, San Francisco, CA (US)

(21) Appl. No.: 17/580,266

(22) Filed: Jan. 20, 2022

Related U.S. Application Data
(63) Continuation-in-part of application No. 17/307,544, filed on May 4, 2021, now abandoned.
(60) Provisional application No. 63/019,704, filed on May 4, 2020.

(51) Int. Cl. G16H 50/20; G16H 20/10; G16H 20/70; G16H 40/67; G16H 80/00; G16H 10/20; H04W 4/02; G08G 1/00

(52) U.S. Cl. CPC G16H 50/20; G16H 20/10; G16H 20/70; G16H 40/67; G08G 1/202; G16H 10/20; H04W 4/023; H04W 4/025; G16H 80/00

(57) ABSTRACT

A system managing a participant health regimen can include any or all of: a computing system; a set of dashboards; a set of models; a user device; a sensor system; one or more supplementary devices; a client application; and/or any other components. A method for managing a participant health regimen includes collecting a set of inputs; determining a participant condition; determining a barrier associated with the participant; and determining and/or triggering an action. Additionally or alternatively, the method can include training and/or retraining any or all of a set of models and/or any other processes.

### CROSS-REFERENCE TO RELATED APPLICATIONS

This application is a continuation-in-part of U.S. application Ser. No. 17/307,544, filed 4 May 2021, which claims the benefit of U.S. Provisional Application No. 63/019,704, filed 4 May 2020, each of which is incorporated in its entirety by this reference.

### TECHNICAL FIELD

This invention relates generally to the health field, and more specifically to a new and useful system and method for managing the health regimens of a set of participants in the health field.

### BACKGROUND

The management of a patient's health or medical condition is a complex process with many players and long timeframes. For medication management specifically, the decision by a patient to take his or her medication is a journey that conventionally starts with a provider visit, an initial prescription, ideally—but often not—the first fill of a medication, and further ideally long-term medication adherence.

With patient adherence to medications and health regimens being especially difficult to monitor and maintain and numerous factors/obstacles getting in the way of adherence such as, for instance: misalignment with providers, lack of long-term motivation, diminishing adherence behavior, other specific behaviors and viewpoints of the patients, and/or any other factors, this can be especially difficult.

Conventional systems and methods attempting to address this focus on low-touch solutions (e.g., automated reminders), which fail to address the complexity of the obstacles to adherence. The inventors have discovered a new and useful behavior-based approach to improve patient health regimen adherence.

### BRIEF DESCRIPTION OF THE FIGURES

FIG. 1 is a schematic of a system for managing a participant health regimen.

FIG. 2 is a schematic of a method for managing a participant health regimen.

FIG. 3 depicts a variation of a set of potential barriers encountered by a participant.

FIG. 4 depicts a variation of a coach dashboard.

FIG. 5 depicts a variation of a participant profile.

FIG. 6 depicts a variation of a coach dashboard and a coach communication determined based on participant information.

FIGS. 7A-7E depict schematic variations of a set of supplementary devices.

FIG. 8 depicts a schematic variation of the system and method for managing a participant health regimen.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview

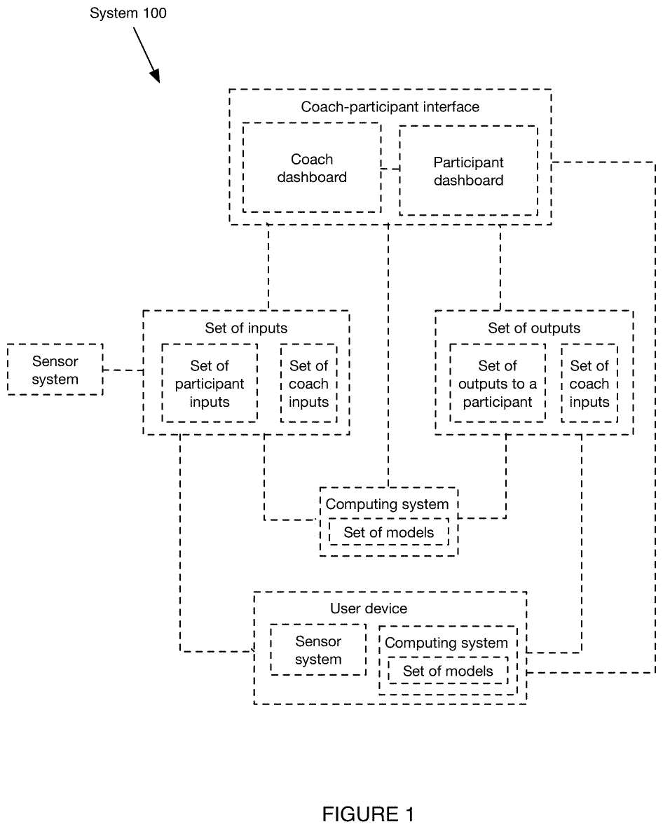

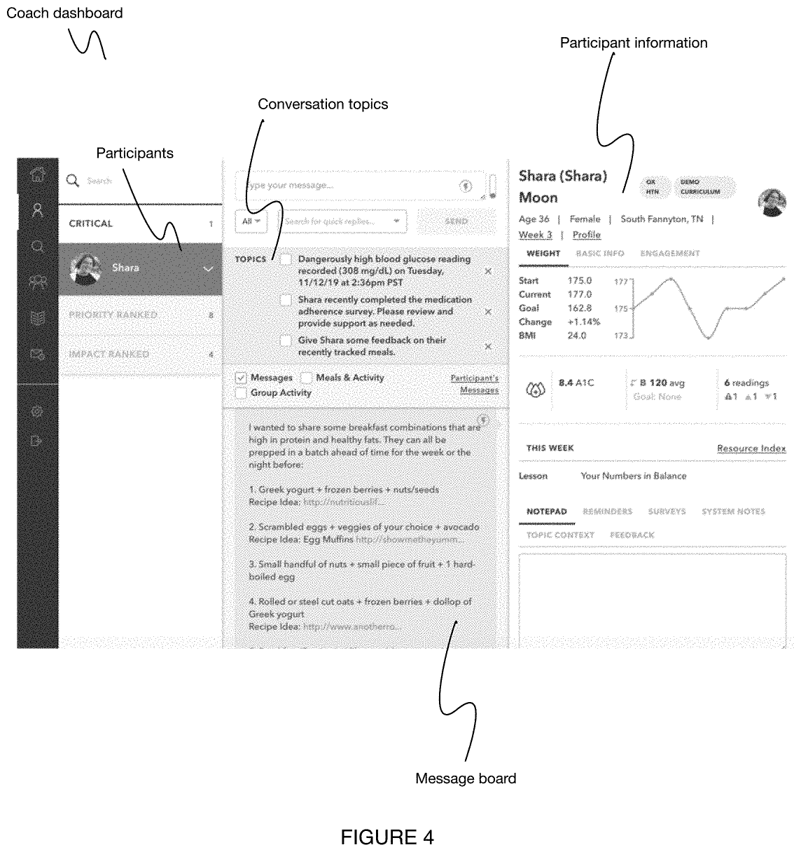

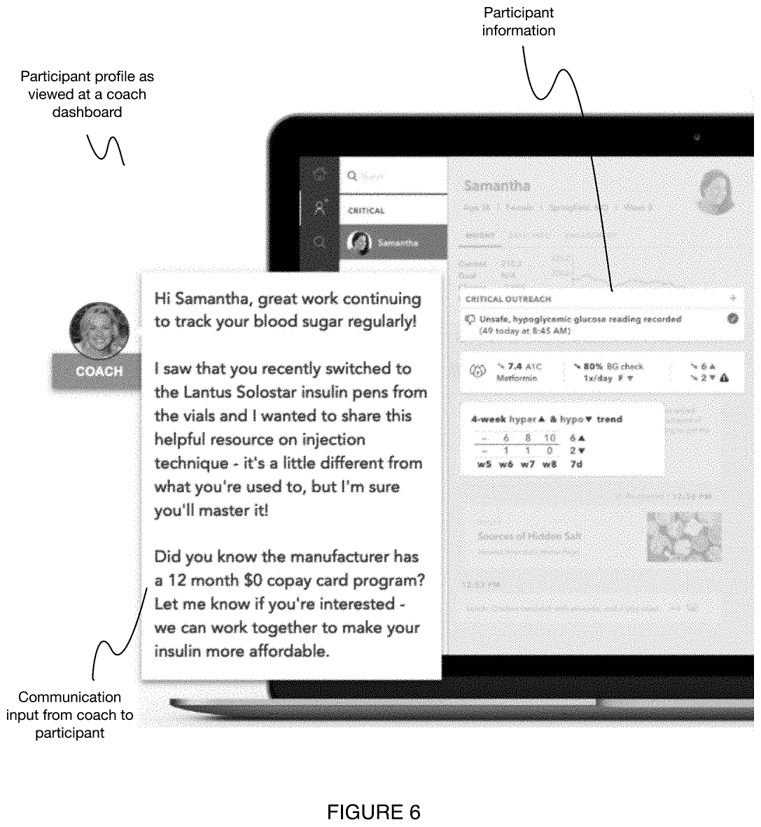

As shown in FIG. 1, a system 100 for managing a participant health regimen can include and/or interface with any or all of: a computing system, a coach-participant interface including a coach dashboard (e.g., as shown in FIG. 4, as shown in FIG. 6) and optionally a participant dashboard; a set of inputs (e.g., from a participant, from a coach, etc.); a set of outputs (e.g., to a participant, to a coach, etc.); a set of models (e.g., to convert the set of inputs into the set of outputs); a user device (e.g., to receive the set of inputs, to display the set of outputs, to display the dashboard, etc.); a sensor system (e.g., weight scale, step counter, accelerometer, etc.) to collect a set of one or more inputs from a set of participants; a supplementary device; a client application (e.g., to execute the dashboards on a user device); and/or any other suitable components.

Further additionally or alternatively, the system can include and/or interface with any or all of the systems, components, embodiments, and/or examples as described in any or all of: U.S. application Ser. No. 15/667,218, filed 2 Aug. 2017, U.S. application Ser. No. 17/195,156, filed 8 Mar. 2021, and U.S. application Ser. No. 17/210,158, filed 23 Mar. 2021, each of which is incorporated herein in its entirety by this reference.

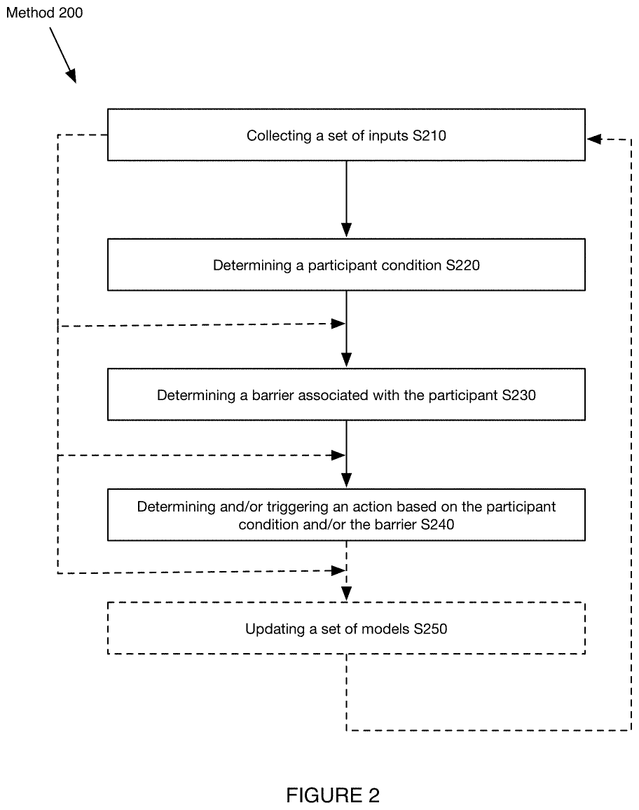

As shown in FIG. 2, a method 200 for managing a participant health regimen includes collecting a set of inputs S210; determining a participant condition S220; determining a barrier associated with the participant S230; and determining and/or triggering an action based on the participant condition and/or the barrier S240. Additionally or alternatively, the method 200 can include training and/or retraining any or all of a set of models; and/or any other suitable processes performed in any suitable order.

Further additionally or alternatively, the method can include and/or interface with any or all of the methods, processes, embodiments, and/or examples as described in any or all of: U.S. application Ser. No. 15/667,218, filed 2 Aug. 2017, U.S. application Ser. No. 17/195,156, filed 8 Mar. 2021, and U.S. application Ser. No. 17/210,158, filed 23 Mar. 2021, each of which is incorporated herein in its entirety by this reference.

The method 200 is preferably performed in accordance with a system 100 as described above, but can additionally or alternatively be performed in accordance with any other suitable system(s).

### 2. Benefits

The system and method for managing a participant health regimen can confer several benefits over current systems and methods.

First, in some variations, the system and/or method confers the benefit of improving the medication adherence and/or adherence to other parts of a health regimen (e.g., exercise, healthy eating, meal planning, reduction in sugar intake, etc.), and subsequently one or more medical and/or health goals (e.g., reducing a hemoglobin A1C level, achieving improved glycemic control, etc.) of a set of participants involved in a remote coaching platform. In a set of specific examples, the system and/or method enables improved medication adherence based on a sustained, personalized, and coordinated efforts through a remote coaching platform to quickly identify non-adherence and address the barriers driving it.

Second, in some variations, the system and/or method confers the benefit of enabling and/or establishing communication between members of a care team (e.g., clinicians, coaches, primary care physicians, pharmacists, etc.) associated with a participant. In specific examples, for instance, the system can aggregate information (e.g., at a coach dashboard, at a remote computing system/remote storage, etc.) accessible by any or all of the care team.

Third, in some variations, the system and/or method confers the benefit of implementing any or all of the features involved in Medication Therapy Management (MTM) in a remote setting. In specific examples, the hindrances (e.g., cost, pharmacist time, etc.) and/or requirements (e.g., multiple chronic conditions, involvement of costly medications, etc.) conventionally encountered in MTM programs are minimized and/or eliminated through a digital, remote implementation of the system and/or method.

Fourth, in some variations, the system and/or method confers the benefit of enabling a robust, comprehensive assessment and monitoring of potential barriers encountered by participants in health regimen adherence, thereby enabling adherence success in vast and varied groups of participants.

Fifth, in some variations, the system and/or method confers the benefit of using learnings and outcomes from prior participants to inform the care and management of future participants. In specific examples, for instance, the method is performed with a set of machine learning models, wherein the machine learning models are trained and retrained based on participant data.

Sixth, a single model that ingests a set of participant inputs (e.g., barriers based on the participant inputs) and directly determines the optimal participant output (e.g., recommendation, message, response, etc.) would be extremely computationally intensive and require large amounts of data, particularly due to the highly irregular nature of the data (e.g., including data related to surveys, health parameters, patient compliance, etc.). In particular, such a model would require statistically significant data across all permutations (including both positive and negative samples) as well as require a sufficient sample set to disambiguate between participants with differing barriers. However, data related to participant health is difficult to acquire and/or simulate, and is thus inherently sparse. The inventors have discovered that, by dividing the participant data based on participant constraints (e.g., selecting the subset of participants having a predetermined value for a given constraint or barrier, selecting the subset of participants having a value for a given constraint that falls within a predetermined range, segmenting the participant population by constraint value, etc.) and determining a model for each constraint division (e.g., population segment), each model can be less computationally intensive (thereby requiring less processing power and/or resulting in a faster computation time without sacrificing the quality of the recommendation), require less memory to store the training data, and require significantly fewer datapoints to train (e.g., reflecting an improvement over conventional neural network systems).

Furthermore, models—especially those predicting or influencing participant behavior—can become outdated quickly as participant behavior shifts (e.g., due to changes in lifestyle, constraints, population-level habits and trends, etc.). By retraining the model based on the participant response (e.g., wherein the model is retrained to output a different result if the participant response is not an expected participant response, or is reinforced to output the same result if the participant response is the desired participant response), the technology can bolster the limited set of training data for each model, and/or keep the model updated and relevant to changes in participant behavior.

Furthermore, variants of the technology can analyze whether a participant is exhibiting aberrant behavioral patterns relative to their own behavioral or response history, and/or relative to their cohort's behavioral or response histories. When aberrant behavioral patterns are detected, the system can: automatically control sensors to sample different measurements (e.g., at a different frequency, a different measurement modality, etc.), automatically control user devices (e.g., participant devices, etc.) to change participant behavior (e.g., guide the user to physical locations different from their cohort, such as different treatment centers or grocery stores; present different interventions than would have been presented to others in the cohort; etc.), apply a different model (e.g., response engine) to the user's data, notify the coach, and/or otherwise respond to the aberrant behavior. By analyzing whether a participant is exhibiting an aberrant behavioral pattern and using the analysis result to operate the sensors, automatically control the participant devices (e.g., to display notifications, control connected devices, adjust participant outputs, display new participant outputs, etc.), and/or guide the participants, this technology can avoid the need for the coach to manually evaluate the behavior of each participant in the group on a continual basis.

Additionally or alternatively, the system and method can confer any other benefit.

### 3. System

As shown in FIG. 1, a system 100 for managing a participant health regimen can include and/or interface with any or all of: a computing system, a coach-participant interface including a coach dashboard (e.g., as shown in FIG. 4, as shown in FIG. 6) and optionally a participant dashboard; a set of inputs (e.g., from a participant, from a coach, etc.); a set of outputs (e.g., to a participant, to a coach, etc.); a set of models (e.g., to convert the set of inputs into the set of outputs); a user device (e.g., to receive the set of inputs, to display the set of outputs, to display the dashboard, etc.); a sensor system (e.g., weight scale, step counter, accelerometer, etc.) to collect a set of one or more inputs from a set of participants; and/or any other suitable components.

Further additionally or alternatively, the system can include and/or interface with any or all of the systems, components, embodiments, and/or examples as described in any or all of: U.S. application Ser. No. 15/667,218, filed 2 Aug. 2017, U.S. application Ser. No. 17/195,156, filed 8 Mar. 2021, and U.S. application Ser. No. 17/210,158, filed 23 Mar. 2021, each of which is incorporated herein in its entirety by this reference.

The system 100 functions to enable a coach and/or other member of a participant's care team to remotely create and/or manage the health regimen of a participant. The system 100 further preferably functions to enable the coach and/or other members to remotely create and/or manage the participant's health regimen in light of a particular set of barriers identified for the participant (e.g., as described below). Additionally or alternatively, the system 100 can function to enable a coach and/or other care team member to assist a participant in achieving one or more of the participant's health goals; enable a coach and/or other care team members to locally manage the participant's health regimen (e.g., with in-person care); establish communication between any or all of: the participant, one or more coaches, one or more members of the participant's care team (e.g., primary care physician, pharmacist, etc.), and/or any other suitable individuals; and/or can perform any other suitable functions.

A health regimen (equivalently referred to herein as a health program) preferably refers to a program in which a participant interacts with one or more coaches (and/or other participants, any or all of a participant's care team, etc.) to achieve one or more health goals and/or adhere to one or more health guidelines (e.g., taking a prescribed medication).

In preferred variations, the health regimen is implemented as part of a digital platform, preferably a digital health platform, wherein coaches and/or other care team members of a participant can provide guidance to and interact with (e.g., through messaging, through the sharing of content, etc.) a set of one or more participants. Additionally or alternatively, a health regimen can be implemented outside of a digital platform (e.g., at a clinic), the digital platform can interface with local care, and/or the health regimen can be otherwise implemented.

A coach refers to an user using the system to engage with and coach a set of one or more participants in achieving one or more goals of the participant. The goals can be any or all of: selected by the participant, recommended by a coach (e.g., in accordance with self-determination theory), recommended by a third party and/or a care team member (e.g., medical professional, employer, family member, etc.), automatically recommended and/or determined (e.g., by one or trained models), predicted, and/or otherwise determined.

Examples of the goals can include, but are not limited to, any or all of: diabetes management (e.g., Type I diabetes, Type II diabetes, prediabetes, etc.), pain management (e.g., for chronic pain, for injury, for joint and muscle pain, for physical therapy and/or rehabilitation, etc.), weight management (e.g., weight loss, weight gain, weight maintenance, etc.), blood pressure management (e.g., blood pressure reduction), mental health management, fitness goals (e.g., run a marathon, increase muscle mass, lose fat, increase cardiovascular exercise, etc.), dietary goals (e.g., adhere to a particular diet, eliminate one or more foods from diet, reduce sugar consumption, reduce carbohydrate consumption, etc.), and/or any other health goals.

Additionally or alternatively, the participant can be associated with non-health-goals (e.g., increase focus, increase job performance, etc.); any number of sub-goals (e.g., lose 10 pounds, maintain a Paleo diet, maintain an A1C level below a predetermined threshold, maintain an A1C level within a healthy range, etc.); and/or any other parameters and/or information.

The coach can refer to either or both of a human agent (equivalently referred to herein as a user) and a non-human agent (e.g., automated agent, robot, chat bot, etc.). For human coaches, the coach can be any or all of: a healthcare professional, a fitness trainer, a nutritionist, a therapist, a medical professional (e.g., doctor, physician, hypertension specialist, physical therapist, occupational therapist, etc.), diabetes care and education specialists, clinical social workers, and/or any other individual affiliated or not affiliated with a health and/or fitness profession.

In preferred variations, a participant is assigned to a human agent who remotely (and/or locally) coaches him or her. Additionally or alternatively, the human agent can be assisted by a non-human agent and/or a set of automated processes, which can assist or replace the human agent in performing certain tasks, such as any or all of: replying to a participant with an auto-generated message (e.g., in absence of the human agent, upon request of the human agent, in an urgent scenario, etc.), auto-filling a message initiated by a human agent, and/or performing any other suitable actions. In preferred variations, the automated content provided by non-human agents and/or automated processes is determined based on trained models (e.g., deep learning models, continuously-updated deep learning models, as described below, etc.), wherein the trained models predict the most impactful response and/or proactive outreach from the human agent based on the observed impact of various strategies in advancing the user goals (e.g., in light of user barriers, for a particular user condition, based on the user's relationship with a coach, etc.). Additionally or alternatively, the automated content can be determined in any other suitable way(s).

Each participant can optionally be associated with a care team, wherein the care team includes agents (human or non-human) involved in the care (e.g., medical care, coaching, etc.) of the participants. As such, the care team preferably includes one or more coaches involved in coaching the participant and optionally one or more clinical agents (e.g., primary care physician, pharmacist, therapist, etc.) involved in the care of the participant. Additionally or alternatively, the care team can include any or all of: a participant's employer and/or insurance agent, other participants, and/or any other suitable agents.

The system 100 can include and/or be configured to interface with any number of computing systems, such as any or all of: a computer, a processor, an analysis engine, a server, and/or any other suitable system(s). The computing system can be any or all of: remote (e.g., cloud computing system), local (e.g., onboard a user device, distributed among multiple user devices, etc.), or any combination of both.

The computing system can optionally implement a set of one or more models (e.g., as described below), which can be used to process any or all of the information collected in S210, such as to determine a user condition, a user barrier, and/or any suitable metrics. Additionally or alternatively, the computing system can implement, store, reference, update (e.g., based on aggregated participant information) and/or otherwise utilize any suitable algorithms, equations, databases, lookup tables, or other information.

The system 100 can include a coach-participant interface, which functions to provide a platform with which a coach can interact with a set of participants. Additionally or alternatively, the coach-participant interface can include a participant dashboard, which functions to provide a platform with which a participant can interact with his or her coach. Further additionally or alternatively, the coach-participant interface can perform any or all of the following functions: providing information to a set of one or more coaches (e.g., informing them of the status of a set of users); providing information to a set of one or more participants (e.g., providing them with information associated with their health regimen(s), providing them with information from their coaches, providing them with resources, etc.); collecting information from a set of participants (e.g., aggregated information with which to update and/or train a set of models, etc.); collecting information from a set of coaches (e.g., with which to train a set of coaches); improve an efficiency of a set of coaches (e.g., through prioritization of coach tasks, through the auto-generation of a set of recommended topics, through easy-to-interpret visualizations, etc.); and/or perform any other suitable function(s).

The dashboard or dashboards of the coach-participant interface are preferably implemented on one or more user devices (e.g., associated with the coach, associated with the participant, etc.), such as through a client application executing on a user device. Additionally or alternatively, the dashboards can be otherwise implemented.

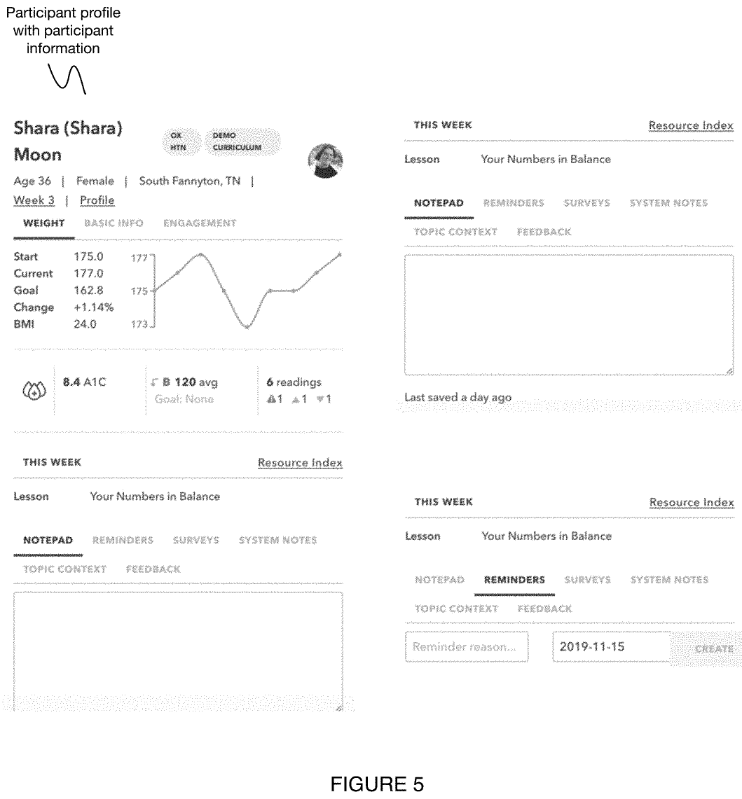

The coach-participant interface includes a coach dashboard (equivalently referred to herein as a coach interface), which functions to provide information associated with a set of one or more participants (e.g., through a participant profile as shown in FIG. 5) to the coach or coaches associated with the set of participants. The dashboard preferably additionally functions to provide a set of tools for the coach (e.g., recommended conversation topics, stored conversation topics, etc.) and to receive inputs from the coach, but can additionally or alternatively perform any other functions.

The system 100 can optionally include and/or interface with any number of user devices (e.g., as described above), which can individually and/or collectively function to: support a dashboard, support a client application (e.g., including the dashboard), receive any or all of a set of inputs (e.g., from a set of client applications, from a set of sensors, etc.), produce any or all of a set of outputs, and/or perform any other suitable functions. Examples of the user device include a tablet, smartphone, mobile phone, laptop, watch, wearable device (e.g., glasses), or any other suitable user device. The user device can include power storage (e.g., a battery), processing systems (e.g., CPU, GPU, memory, etc.), user outputs (e.g., display, speaker, vibration mechanism, etc.), user inputs (e.g., a keyboard, touchscreen, microphone, etc.), a location system (e.g., a GPS system), sensors (e.g., optical sensors, such as light sensors and cameras, orientation sensors, such as accelerometers, gyroscopes, and altimeters, audio sensors, such as microphones, magnetometers, etc.), data communication system (e.g., a WiFi transceiver(s), Bluetooth transceiver(s), cellular transceiver(s), etc.), or any other suitable component. The user device is preferably in communication with (e.g., wireless communication, Wifi communication, Bluetooth communication, radio communication, wired communication, etc.) one or more computing systems, but can additionally or alternatively be in communication with any other components (e.g., supplementary device as described below), absent of communication with other components, and/or otherwise configured. Further additionally or alternatively, any or all of the computing system can be arranged onboard the user device.

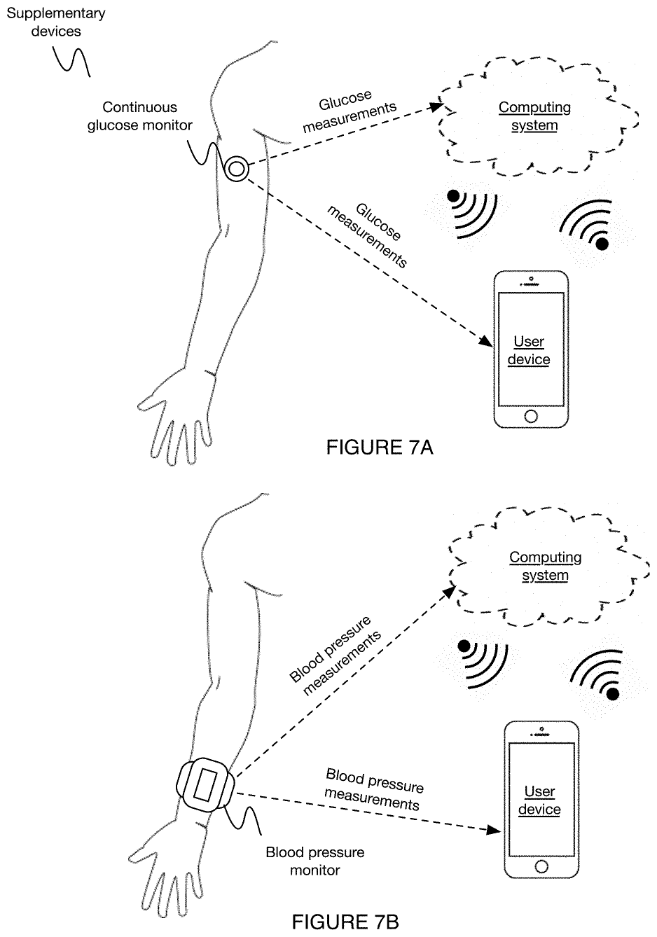

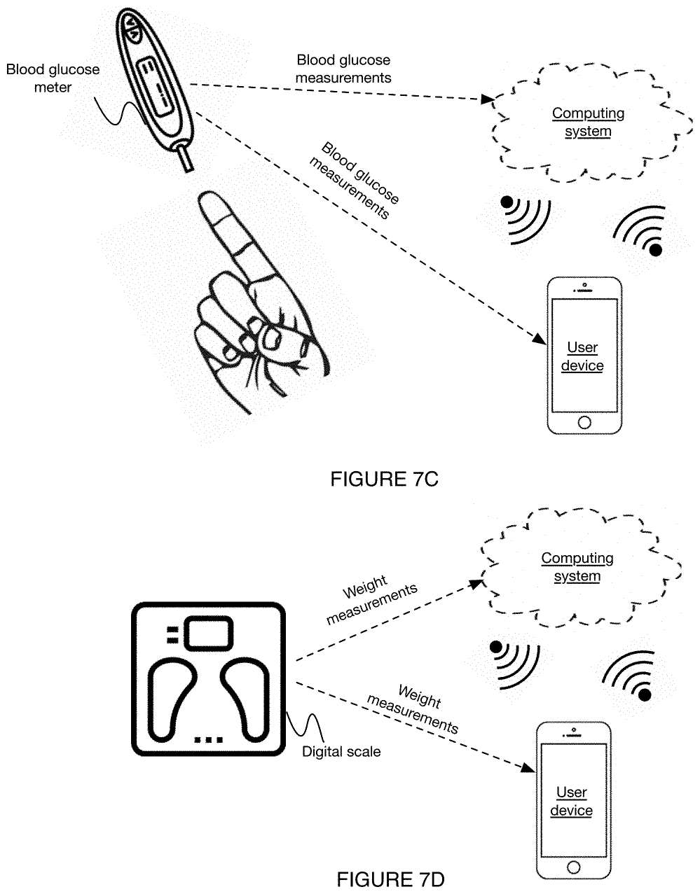

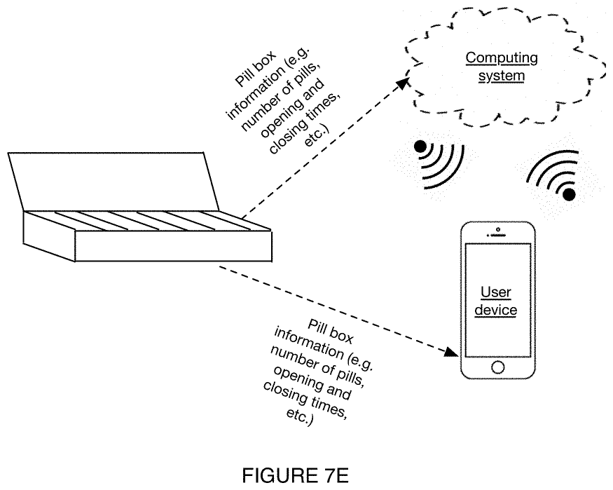

The system can optionally include and/or interface with any number of supplementary devices (e.g., as shown in FIGS. 7A-7E), wherein the supplementary devices can function to provide additional inputs (e.g., sensor information, biological information, biometric signals, etc.), process inputs, be used in the care of the participant and/or adherence to a health regimen, and/or can perform any other functions. The supplementary devices can include any or all of: medical devices, health devices (e.g., consumer health devices), sensors, fitness equipment (e.g., fitness trackers, workout equipment, sports accessories such as balls and/or tennis rackets, etc.), and/or any other devices. The supplementary device is preferably in communication with (e.g., wireless communication, Wifi communication, Bluetooth communication, radio communication, wired communication, etc.) one or more computing systems, but can additionally or alternatively be in communication with any other components (e.g., user device as described above), absent of communication with other components, and/or otherwise configured. Further additionally or alternatively, any or all of the computing system can be arranged onboard the supplementary device.

In a preferred set of variations, for instance, any or all of the supplementary devices can be in wireless communication with a remote computing system, such that the supplementary devices can transmit measurements (e.g., weight values, AIC levels, etc.) and/or other parameters to the remote computing system.

Examples of supplementary devices include a pill box (e.g., with sensors to detect when it's been open and/or closed, to detect what pills are taken and when, etc.), a scale for measuring a weight of the user (e.g., and in wireless communication with a computing system), a medical device and/or diagnostic device (e.g., blood glucose meter, continuous glucose monitor [CGM] sensor, blood pressure monitor, etc.), a fitness tracker (e.g., step counter, motion detector, etc.), and/or any other devices. In a first set of specific examples, for participants taking part in a diabetes management health regimen, the participant can interface with a blood glucose meter and a CGM sensor which can be adhered to and/or inserted into a skin surface of the user (e.g., to continuously detect and transmit a set of glucose measurements such as an AIC level). In a second set of specific examples, for participants taking part in a blood pressure management health regimen, the participant can interface with a digital scale (e.g., which wirelessly transmits weight measurements to a computing system) and a blood pressure monitor (e.g., which wirelessly transmits blood pressure measurements to a computing system). In a third set of specific examples, for participants taking part in a prediabetes and/or weight management health regimen, the participant can interface with a digital scale.

Additionally or alternatively, the supplementary devices can include and/or interface one or more 3^{rd} party client applications which can collect information from the supplementary devices (e.g., and transmit to the client application), process information from the supplementary devices, and/or otherwise provide information to a computing system and/or client application of the system.

The system 100 can include and/or interface with any number of sensor systems. The sensor systems can be part of a user device (e.g., accelerometer, location sensor, camera, microphone, etc.), part of a supplementary device (e.g., scale, pedometer, step counter, pill box, etc.), associated with a medical device and/or a diagnostic system (e.g., a glucose monitor, blood pressure cuff, insulin pen, etc.), and/or part of any other suitable components. In some variations, for instance, one or more participant inputs are received from the sensor system. In specific examples, the sensor system is part of (e.g., onboard) any or all of: a connected scale for monitoring of a user's weight, a fitness tracker (e.g., step counter), a sleep tracker (e.g., as part of a smart watch), a medical device (e.g., glucose monitor, blood pressure cuff, etc.), and/or any other suitable sensor systems. Additionally or alternatively, any or all of the sensor systems and/or supplementary devices described in U.S. application Ser. No. 15/667,218, filed 2 Aug. 2017, U.S. application Ser. No. 17/195,156, filed 8 Mar. 2021, and U.S. application Ser. No. 17/210,158, filed 23 Mar. 2021, each of which is incorporated herein in its entirety by this reference, can be included.

### 4. Method

The method 200 functions to assist a participant in managing a medical condition and/or achieving a health goal or other goal. In preferred variations, the method 200 functions to enable a coach and/or any other members of a participant care team to remotely monitor and/or guide a participant in the self-management of a medical condition and/or the maintenance of a health regimen based on remotely-determined participant barriers. Additionally or alternatively, the method 200 can function to enable communication among multiple coaches and/or members of a participant care team, such as between any or all of: one or more coaches, a primary care physician of the participant, a pharmacist associated with the participant, a therapist associated with the participant, and/or any other suitable entities and individuals.

In some variations, the method 200 is at least partially performed in accordance with self-management theory. In preferred variations of the method 200, for instance, each of the participants is involved in the selection of and his or her progress toward a set of health goals.

Additionally or alternatively, the method 200 can be at least partially performed in accordance with medication therapy management (MTM). In preferred variations of the method 200, for instance, remote coaching and/or communication among care team members enables robust remote management of a participant's medical conditions.

The method 200 is further preferably at least partially performed based on coach knowledge of a participant's barriers to medication adherence. In preferred variations of the method 200, for instance, through the determination by a coach of a participant's particular barriers to adherence, the coach can enable a participant to effectively self-manage his or her conditions. In specific examples, the coach can refrain from solely reminder-based approaches, such as sending reminders to the user to adhere to his or her regimen and/or program (e.g., take medication, exercise, meal plan, diet, take weight measurements, wear a glucose monitor, take a blood glucose measurements, do rehabilitation exercises, etc.). Additionally or alternatively, the coach can

Additionally or alternatively, the method 200 can otherwise function and/or be otherwise performed.

Any or all of the method can be performed with a set of models, algorithms, and/or tools, such as any or all of those described below. In some variations, for instance, any or all of the following are performed with a set of models, algorithms, and/or tools: determining a goal of the user, determining a barrier of the user, determining and/or triggering an action, transmitting a set of surveys to the participant, and/or any other process. The set of models preferably includes one or more trained models (e.g., artificial intelligence [AI] models, machine learning models, deep learning models, neural networks, etc.), but can additionally or alternatively include rule-based models and/or algorithms, untrained models, and/or any other tools.

The set of models can optionally implement predictive modeling, which can function, for instance, to predict the incremental impact of an action on the participant's behavior (e.g., in light of a particular barrier). This can be implemented, for instance, to determine actions that would result in the participant doing better (e.g., moving closer to achieving goals, exhibiting better engagement with coach, etc.). In some variations, for instance, the predictive modeling technique includes uplift modeling (equivalently referred to herein as incremental building, true lift modeling, net modeling, etc.). Additionally or alternatively, any other predictive modeling or other type of modeling can be used.

The set of models can additionally or alternatively implement regression modeling, which functions to predict multiple variables, such as multiple correlated dependent variables. In preferred variations, the set of regression models includes a set of multivariate linear regression models, with the variables including any number and type of metrics (markers) associated with the success of a participant. These can include an engagement level of a participant with a coach, which can be measured through any or all of: participant's usage of a client application, time spent interacting with the participant dashboard, number of messages exchanged with a coach, length of messages exchanged with a coach, time between interactions with a coach, frequency of interactions with a coach, and/or any other suitable parameters. In a first set of variations, for instance, one or more uplift models are used which implement one or more regression techniques (e.g., linear regression, non-linear regression, etc.). Additionally or alternatively, regression modeling can be used elsewhere, the uplift models and/or other predictive models can be otherwise implemented, and/or any other suitable models can be used.

The set of models can further additionally or alternatively include one or more topic models which implement a topic modeling approach (e.g., unsupervised topic modeling, supervised topic classification, Correlation Explanation [CorEx], etc.) to assess one or more features (e.g., tone, emotional content, bonding, rapport, etc.) of messages exchanged between the coach and a participant. This can then optionally be used, for instance, to determine which participants the coach as the best rapport with (e.g., best connection, best engagement, etc.), as the inventors have discovered that participants with the best rapport benefit the most from coaches reaching out to them. In specific examples, for instance, one or more scores (e.g., impact score) can take this into account to prioritize coaches reaching out to participants with whom they have the best rapport. Additionally or alternatively, participants can be otherwise prioritized and/or incentivized, and/or the topic modeling can be otherwise suitably implemented, such as to determine which topics the coach has covered in his or her conversations with the participant. In specific examples, a CorEx model is used to score the topic and/or sentiment of messages from a coach to a participant based on the raw text of the messages.

The set of models can further additionally or alternatively include models with which to determine a likelihood that a participant will respond favorably to a notification (e.g., reminder) from a coach at a particular moment in time. This can be implemented, for instance, with one or more reinforcement learning models, which can determine a favorability likelihood score based on the participant's clickstream data (e.g., historical information showing his or her clickstream in response to a notification) and/or any other suitable information. Additionally or alternatively, any other suitable models can be used.

The set of models can further additionally or alternatively include models with which to determine one or more scores quantifying a predicted outcome associated with a participant, such as his or her progress (e.g., actual progress, predicted progress, etc.) in reaching a goal, a relative predicted change in one or more conditions (e.g., health conditions, diabetes, high blood pressure, etc.) or outcomes, and/or any other suitable metrics. The set of models for determining a predicted outcome score preferably include one or more linear models (e.g., generalized linear model), but can additionally or alternatively include one or more nonlinear models, other regression models, and/or any other suitable models. In specific examples, these models take into account participant demographic information, participant behavior information, and historical participant outcome information, but can additionally or alternatively take into account any other inputs.

In some variations, the method functions to provide a robust and scalable tool for coaches to help optimize his or her interactions with participants without requiring a large amount of training data. In specific examples, for instance, the system and/or method optimizes these interactions with a set of trained uplift models which require a relatively small amount of data for training (e.g., relative to reinforcement learning (RL) and/or other types of machine learning) and do not require to be manually programmed and updated as in rule-based approaches.

Any or all of the method 200 can be performed automatically, manually, or any combination.

Additionally or alternatively, the method 200 can be otherwise implemented.

### 4.1 Method—Receiving a Set of Inputs S210

The method 200 includes receiving a set of inputs S210, which functions to collect information with which to determine (e.g., automatically, with coach input, etc.) any or all of: a condition (e.g., medical condition, health condition, etc.) of the participant, a barrier associated with the participant (e.g., barrier to adherence), a goal of the participant, one or more metrics associated with the participant (e.g., performance metrics, progress metrics, goal achievement metrics, etc.), lifestyle information associated with the participant (e.g., fitness/activity level, meals consumed, mobility information, etc.), sensor information (e.g., location information, motion information, etc.), demographic information (e.g., age, gender, ethnicity, weight, height, body mass index, etc.), habit and/or preference information (e.g., based on clickstream data, shopping habits, etc.), progress and/or success of the participant (e.g., for updating a set of models, etc.), and/or any other suitable information.

S210 is preferably performed initially in the method 200, but can additionally or alternatively be performed in response to other processes of the method 200, as part of any other processes of the method 200, in parallel with any other processes of the method 200, multiple times during the method 200 (e.g., continuously, at a predetermined frequency, at random intervals, etc.), in response to a trigger, and/or at any other suitable times.

The set of inputs preferably includes participant information, but S210 can additionally or alternatively include collecting any other suitable information and/or inputs, such as any or all of: coach information and/or inputs (e.g., messaging information of a coach), care team information and/or inputs, and/or any other suitable information.

The set of inputs can be received from any or all of: a set of one or more participants, a set of one or more coaches, a computing system (e.g., as generated by the set of models), external (e.g., 3^{rd} party) information sources (e.g., census information, online information, medical records such as a participant's medical history, clinical records, etc.), a user device and/or client applications (e.g., a meal tracker client application, a fitness tracker client application, sleep tracker client application, etc.), external devices (e.g., smart watch), supplementary devices (e.g., medical devices), and/or any other suitable information sources.

The participant information can be received at any or all of: one or more user devices, a computing system (e.g., a remote computing system, computing system of a user device, etc.) and/or server (e.g., remote server, cloud-based server, etc.), a dashboard (e.g., coach dashboard, participant dashboard, etc.), and/or any other suitable locations.

In some variations, at least a portion of the participant information is collected without participant intervention (e.g., passively collected from any or all of: medical records, connected sensors, user devices, care team members and associated databases, etc.), but can additionally or alternatively be collected based on user input (e.g., filling out a set of surveys, manually inputting information into a participant profile, etc.).

Any or all of the participant information can be collected continuously (e.g., at a predetermined frequency, upon being input into a participant profile by the participant, upon being input into a dashboard by a coach and/or a participant, at an intermittent frequency, etc.). Additionally or alternatively, any or all of the participant information can be collected once (e.g., participant demographic information), in response to a trigger, upon being updated, and/or at any other suitable times. In preferred variations, the participant information is collected throughout the method 200, further preferably throughout the duration of the participant's participation in the health program.

In preferred variations, participant information is collected continuously throughout the method 200 and throughout the duration of the participant's participation in the health program (e.g., upon entering of information from any or all of: the participant, coaches, and care team members), wherein one or more interactions and/or actions described further in the method 200 can be determined and/or updated based on the updated information. In specific examples, for instance, coaches and/or care team members associated with the participant can monitor (e.g., flag) and/or trigger an adjustment of medication based on the information, as medication needs can change based on numerous factors, such as any or all of: age, lifestyle, weight (e.g., as determined based on weight measurements from a smart scale in communication with the coach dashboard), the addition of a new medication, the development of the medication condition, a participant's mental health (e.g., as assessed by one or more models, as assessed through communications with a coach, etc.), and/or any other information.

The participant information can be any or all of: provided by the participant, provided by an external source (e.g., healthcare facility visited by the participant, sampled at a sensor system of a user device and/or external device, etc.), provided by a coach and/or a care team member (e.g., received from a primary care physician of the participant, received from a pharmacist associated with the participant, etc.), determined by a computing system (e.g., through a set of models), and/or any combination of both (e.g., provided by a participant and processed at a computing system).

The participant information preferably includes information associated with a medical condition of the participant, such as diagnostic information (e.g., results of one or more medical tests), routine testing information (e.g., glucose levels, hemoglobin levels, etc.), medication information (e.g., types of medication being taken by the participant; medication adherence information such as any or all of: time of medication consumption, evidence of medication consumption, tracking of participant's medication supply, amount of medication in participant's supply, etc.; etc.), prescription information (e.g., medication prescribed to participant, record of participant picking up prescription from a pharmacy, refill guidelines associated with medication, etc.), and/or any other suitable medical information. In preferred variations, the medical information is collected from any or all of: sensors, such as those associated with a medical device (e.g., pill box, insulin pen such as a number of uses and/or an amount of insulin remaining in the insulin pen, blood pressure cuff, hemoglobin sensor, blood sugar test, random blood sugar test, fasting blood sugar test, oral glucose tolerance test, inhaler, etc.); a user device associated with the participant (e.g., motion sensors onboard the user device to collect mobility information, location sensors onboard the user device to collect location information such as evidence of a user's trip to a pharmacy, image sensors to capture photos/videos of participant taking medication, etc.); a device and/or database associated with a care team member of the participant (e.g., EHR/EMR records, other medical record databases, pharmacy records, clinical notes from primary care physician, etc.); and/or any other suitable information sources.

The set of participant information can additionally or alternatively include demographic information associated with the participants, such as any or all of: age, sex, and race. Additionally or alternatively, the set of features and/or parameters can include lifestyle information (e.g., occupation, activity/fitness level, schedule, etc.), other inputs, body composition (e.g., current weight, height, measurements, body mass index [BMI], ectomorph vs. endomorph vs. mesomorph, etc.) or other body parameters, medical or biometric information (e.g., blood pressure, glucose level, presence of a medical condition, etc.), income level, socioeconomic conditions associated with the user, social determinants of health associated with the user (e.g., local availability of health food options, local availability of fitness opportunities such as gyms and/or outdoor spaces, income level, education level, marital status, parental status, residential conditions, etc.), geographic/location information of user (e.g., location of residence, zip code of residence, proximity to a grocery store with healthy food options, proximity to gym, etc.), employment information (e.g., employed vs. not, hours spent working vs. amount of free time, number of jobs, etc.), any existing health conditions (e.g., cancer, high blood pressure, low blood pressure, Type-I diabetes, Type-II diabetes, etc.), and/or any other suitable information.

In some examples, for instance, location information (e.g., zip code of residence) of the user is used to determine a quality of the social determinants in that location, wherein the quality of the social determinants can be used (e.g., by a coach, by one or more models, etc.) in the determination of a barrier and/or in an action (e.g., as described below).

In a specific example, for instance, it can be detected (e.g., based on zip code or other location information) that a participant lives in a food desert, wherein a barrier (e.g., logistics, cost, etc.) can be selected at least in part based on this.

In some variations, any or all of the medication information is collected in combination with a medication database (e.g., Lexicomp database), which can function to ensure that accurate and/or interpretable information (e.g., without typographical errors, with easy access to all associated information, etc.) is collected.

The participant information can optionally include one or more clinical outcomes (e.g., diabetes, depression risk, medical records, etc.). In some variations, clinical outcomes are received in response to a user providing consent for the system to access clinical records (e.g., from a healthcare facility, from a medical records database, etc.). Additionally or alternatively, the clinical outcomes can be otherwise received (e.g., directly from the participant, etc.).

The participant information can optionally include participant preferences, such as goals and motivations (e.g., look better, lose weight, complete a particular fitness activity, increase strength, increase endurance, reduce severity of a medical condition, manage diabetes, manage a hemoglobin level, etc.). The participant preferences can be determined based on any or all of: clickstream data (e.g., within a client application containing the participant dashboard, within external client applications, on the internet, etc.), survey information, direct participant inputs, coach inputs, care team member inputs, and/or any other suitable sources.

S210 preferably includes administering and collecting information from each participant through a set of one or more surveys, which are preferably used subsequently in the method 200 in the determination of one or more adherence barriers associated with the participant. Additionally or alternatively, the survey results can be otherwise used. S210 can optionally include determining a timing associated with any or all of the surveys. This can be important in the preferred remote coaching embodiments of the method 200, as the timing can have an effect on trust and engagement levels of the participant with respect to any or all of the program/health regimen, coach(es), and/or any other aspects. In some cases, it has been shown, for instance, that asking for information deemed personal to a participant in a survey can cause immediate disengagement due to a lack of trust felt by the participant. Waiting a threshold time period (e.g., 4 weeks, 3 weeks, 5 weeks, greater than a day and less than 6 weeks, greater than 2 weeks and less than 8 weeks, less than 4 weeks, greater than 4 weeks, etc.) can enable communication between participants and coaches to be adequately established prior to administering a survey.

In some variations, for instance, a set of multiple surveys is administered to the participant, wherein a time delay is enforced between subsequent surveys. The time delay can be determined based on any or all of: a minimum threshold (e.g., wherein the time delay is equal to or exceeds a predetermined minimum threshold), a maximum threshold (e.g., wherein the time delay is below the maximum threshold, wherein the time delay is above the minimum threshold and below the maximum threshold, etc.), a time delay configured for a particular participant (e.g., based on participant preferences, based on coach input, etc.), a dynamically determined delay (e.g., based on a trained model, based on a decision tree, based on historical information for the participant, based on historical information for other participants, etc.) any combination, and/or any other delays. For a set of multiple surveys including more than 2 surveys, the time delays can be the same between surveys, different between surveys (e.g., increasing with subsequent surveys, decreasing with subsequent surveys, etc.), and/or otherwise configured.

The survey timing can be set for all participants, or additionally or alternatively determined based on any or all of: a medical condition associated with the participant, demographic information associated with the participant, historical information associated with the participant (e.g., time since joining the program, time since last survey, etc.), coach preference, determined based on one or more models, and/or otherwise determined.

In preferred variations, a set of surveys (e.g., questionnaires) and/or other self-report tools are used to identify medication-taking barriers (e.g., as described below).

The set of surveys are preferably administered through a remote platform as described above (e.g., a virtual disease management platform), which can confer several advantages, such as any or all of: easily administering surveys to each of a set of participants (e.g., all participants) at multiple touch points throughout their health care journey; delivering surveys anonymously through the platform (e.g., which can reduce response bias); and/or conferring any other suitable benefits.

The participant information can include lifestyle data, such as any or all of: food consumed by the participant (e.g., tracked meals, tracked calories, photos of food, etc.), exercise performed by the participant (e.g., as input by the participant, as detected through one or more sensors such as a pedometer, etc.), and/or any other suitable information. Additionally or alternatively, participant information (e.g., participant preferences) can be determined based on clickstream data (e.g., at the client application, at a 3^{rd} party client application, etc.) and/or otherwise determined.

In a first set of variations, a set of inputs is initially collected in S210 for each of a set of participants (e.g., at onboarding), wherein the set of inputs includes information about a participant's current health status, a care plan (e.g., blood pressure goal as defined by their doctor), prescribed medications, as well as personal goals and known barriers to achieving those goals. Throughout the participant's participation in the remote coaching program, additional inputs are continuously collected, such as any or all of: updates to the previous information; sensor data associated with each participant (e.g., weight of the patient, biometric parameter of the participant, etc.); coach-participant engagement information associated with each participant (e.g., number of messages exchanged between the coach and the participant, content of message exchanged between the coach and the participant, etc.); survey results from one or more participants; and/or any other suitable information.

The set of inputs can additionally or alternatively include any other information, such as any or all of: historical participant information (e.g., for assigning a participant into a group); coach inputs (e.g., message information, coach assignment of a goal and/or a barrier to the participant, etc.); messaging information (e.g., content of messages exchanged between a participant and a coach, frequency of messages exchanged between a participant and a coach, response time of the participant, length of messages, etc.); and/or any other suitable inputs.

In a first variation, S210 includes collecting participant inputs from a client application and a set of sensor systems, wherein the participant inputs can be used in any or all of the following processes of the method.

In a first specific example, the participant inputs include messaging information associated with messages exchanged between the participant and one or more coaches, survey information from the participant, sensor information from a user device (e.g., location information of the participant, motion information of the participant, etc.), and sensor information from a supplementary device (e.g., glucose levels, weight values, etc.).

In a second variation, S210 can additionally or alternatively include collecting information from a set of 3^{rd} party client applications and/or databases.

Additionally or alternatively, the set of inputs can include coach inputs, inputs from other participants, 3^{rd} party inputs, and/or any other inputs.

Further additionally or alternatively, S210 can include any other processes and/or be otherwise performed.

### 4.2 Method: Determining a Participant Condition Based on the Participant Information S220

The method can optionally include determining a participant condition based on the participant information S220, which functions to inform the coaching of the participant and make it most effective for that particular condition.

S220 is preferably performed in response to S210, but can additionally or alternatively be performed prior to S210, independently and/or in absence of S210, in response to other processes of the method 200, as part of any other processes of the method 200, in parallel with any other processes of the method 200, multiple times during the method 200 (e.g., continuously, at a predetermined frequency, at random intervals, etc.), in response to a trigger, and/or at any other suitable times.

S220 is further preferably performed after at least a first instance of S210, such as after initial participant information (e.g., demographic information, information from medical records, etc.) is collected. S220 can optionally be performed multiple times throughout the method 200, wherein the user condition is monitored and/or updated based on new participant information and optionally any other information (e.g., coach feedback, clinician feedback, etc.).

The participant condition can be any or all of: a health condition (e.g., medical condition, known disease, syndrome, illness, etc.), a general health and/or fitness assessment (e.g., fit, healthy, active, sedentary, high risk, moderate risk, low risk, overweight, underweight, etc.); and/or any other characterization of the participant which functions to enable a coach to target his or her coaching (e.g., through communications, resources sent, check-ins, etc.) to the participant condition. In preferred variations, for instance, health-condition-specific content is layered into the core lessons of the coaches, personalizing the experience and embedding the participant's medications and/or health regimen tasks (e.g., exercising, taking blood glucose measurements, weighing himself or herself, etc.) within the rest of the condition, lifestyle, and behavioral discussions.

Additionally or alternatively, the participant condition can be predetermined (e.g., assigned to the participant upon onboarding), determined based on information other than participant information, determined based on a set of one or more surveys, received from a physician associated with the participant, and/or otherwise determined.

For participants associated with one or more health conditions, the health condition(s) can be any or all of: a chronic condition, a non-chronic condition, and/or any other suitable condition. In some variations, the medical condition includes one or more of: diabetes (e.g., prediabetes, Type I diabetes, Type II diabetes, etc.), hypertension, dyslipidemia, high cholesterol, anxiety, depression, asthma, a heart condition, and/or any other suitable condition.

The participant condition can be determined based on any or all of: a set of models (e.g., deep learning models at a remote computing system) and/or algorithms, a lookup table or database, inputs from the participant's care team (e.g., primary care physician) and/or medical records, and/or based on any other suitable information.

S220 can optionally additionally or alternatively include determining a goal (e.g., as described above) associated with one or more users. The goal is preferably determined based on and/or associated with a participant condition (e.g., related to management and/or improvement of a health condition), but can additionally or alternatively be otherwise determined.

In a first set of specific examples, a goal of the participant is determined automatically, such as with a trained model based on the set of inputs and/or the participant condition. In a second set of specific examples, a goal of the participant is selected by the participant and/or the coach. In a third set of specific examples, a goal of the user is determined based on a participant condition and with a lookup table and/or decision tree. Additionally or alternatively, goals of the participant can be otherwise determined.

S220 can optionally include assigning the participant to a cohort of participants (e.g., among all participants in the program, among all participants associated with a coach, etc.), wherein the cohort assignment can be used in any or all of: the selection of one or models, algorithms, rules, or lookup tables (e.g., to select a set of materials to send to the participant, to inform coach communications, etc.); subsequent cohort assignments (e.g., barrier cohorts); and/or can be used in any other suitable way(s).

In a first variant, for instance, one or more models can be specifically trained for a particular participant condition (e.g., based on historical information from participants associated with that particular condition) and/or participant barrier, wherein the participant is assigned to a group (e.g., based on a shared condition, shared barrier, etc.) and analyzed with the model(s) associated with that group.

In an example of the first variant, the participants are divided into groups (e.g., training groups) based on participant barriers (e.g., barriers, barrier values, ranges of barrier values, etc.). A model associated with a set of barriers (e.g., one or more barriers) is then trained on data (e.g., previous received data including inputs mapped to outputs, successful and/or unsuccessful interventions, and/or responses for each user) associated with the participants in a training group, wherein participants in the training group are associated with the same set of barriers as the model (e.g., where the set of barriers associated with the model can include a range of barrier values). For a given participant, the model sharing a set of barriers with the participant (e.g., the barriers for the given participant match the barriers associated with the model, fall within a range prescribed by the model barriers, etc.) can be selected to analyze the participant (e.g., the participant's data), generate an output, control a device, and/or for use in any other action. The given participant can be a part of the group used to train the selected model and/or be separate from the group.

In a second variant, the inputs for a participant assigned to a cohort can be analyzed and compared to the cohort (e.g., to other participants in the cohort, to a set of inputs aggregated from the cohort, etc.) to determine whether the user is exhibiting aberrant behavioral patterns (e.g., by a statistically significant margin, exceeding behavioral pattern thresholds, etc.). Examples of aberrant behavioral patterns include: outlier biometric parameter measurements and/or other inputs, significantly fewer user inputs, deviating user responses, unexpected deviations in data trends, and/or any other aberrant datapoint and/or pattern. In response to a determination that the user is exhibiting aberrant behavioral patterns: one or more devices can be automatically and/or manually controlled (e.g., sampling different parameters, collecting additional sensor measurements, recalibrating a sensor, sending a notification to the user and/or coach, collecting additional user inputs, etc.), one or more outputs can be determined (e.g., adjusting a previously determined output, determining a new output), the user can be re-grouped into a new cohort, a different model can bused to determine outputs (e.g., interventions) for the user (e.g., a model trained on data from participants sharing the aberrant behavior), and/or any other action can be performed.

Additionally or alternatively, the groups can be determined based on any or all of: a barrier of the participant, a goal of the participant, and/or any other information.

In a first variation, S220 includes determining a participant condition based on the participant's communication of his or her condition to a coach.

In a second variation, S220 includes determining a participant condition based on clinical records and/or input from a clinician associated with the participant.

In a third variation, S220 includes automatically determining a participant condition, such as with a trained model, and optionally any or all of the set of inputs.

Additionally or alternatively, S220 can include any other processes and/or be otherwise suitably performed.

4.3 Method: Determining a Barrier Associated with the Participant Based on the Participant Information and/or the Participant Condition S230

The method includes determining a barrier (e.g., a constraint) associated with the participant based on the participant information and/or the participant condition S230, which functions to further specialize the coaching of the participant and enable the participant to most effectively self-manage or partially self-manage his or her participant condition.

S230 is preferably performed in response to S220, but can additionally or alternatively be performed prior to S220, independently and/or in absence of S220, in response to other processes of the method 200, as part of any other processes of the method 200, in parallel with any other processes of the method 200, multiple times during the method 200 (e.g., continuously, at a predetermined frequency, at random intervals, etc.), in response to a trigger, and/or at any other suitable times.

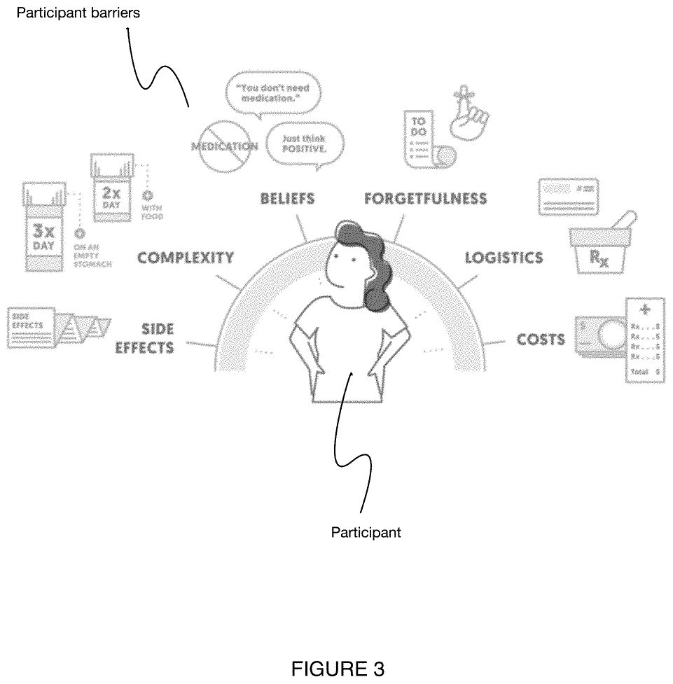

The barrier is preferably related to adherence to one or more aspects of maintaining a proper health regimen for the participant condition, further preferably to adherence of one or more medications prescribed to the participant, but can additionally include barriers to adherence of other aspects of a health regimen (e.g., exercising at a predetermined frequency, maintaining a particular weight, etc.), and/or any other barriers which may prevent a participant from reaching his or her health goals. Barriers to adherence are common and can be varied among different participants, as medication-taking behavior is complex and multifactorial: the decision to take-or not take-medication can involve any combination of patient, provider, and health care system-related factors, such as, but not limited to, any or all of (e.g., as shown in FIG. 3): health beliefs, medication complexity, side effects, forgetfulness, logistics, and cost.

Barriers to medication adherence can include any number of provider-related factors, as providers can often overlook medication nonadherence. In some cases, overly complicated medical regimens are prescribed with little time to explain the benefits and adverse effects of new medications effectively. Further, in many cases, the importance of medication adherence is not discussed, nor is the financial burden on the patient considered.

Additionally or alternatively, barriers to health regimen adherence (e.g., medication adherence) can include any number of participant-related factors (equivalently referred to herein as patient-related factors). It has been discovered, for instance, that participants (equivalently referred to herein as patients) recall and comprehend as little as 50% of what their physicians tell them, making them far less likely to possess critical self-management skills or engage in the continuous decision-making required to adhere to medications and a care plan. Further, any or all of the financial status, mobility, personal beliefs surrounding medication, and/or other aspects of each individual participant can contribute to barriers to health regimen adherence.

In specific examples, the barrier(s) associated with each participant are selected from the following barriers: health beliefs of the participant (e.g., medication is not necessary, propensity toward natural remedies, belief that the condition can be treated without medicine, etc.), cost (e.g., financial limitations of participant, particular insurance coverage, participant is associated with a set of social determinants, etc.), complexity (e.g., of medication management, of the medication itself, etc.), forgetfulness (e.g., in taking medication on a daily basis), logistics (e.g., of obtaining the medication/filling prescriptions, distance to a pharmacy, distance to a gym, distance to a grocery store and/or health foods store, distance to a fast food restaurant, participant mobility, participant is associated with a set of social determinants, participant does not have a driver's license and/or vehicle, etc.), and side effects (e.g., of the particular medication prescribed, of medication in general, etc.). Additionally or alternatively, any other suitable barriers can be identified for a participant.

The barrier(s) associated with each participant are preferably at least automatically determined (e.g., at a remote computing system including a memory and a processing system) based on participant information collected through a set of surveys (e.g., as described above). Additionally or alternatively, any other suitable information described above (e.g., historical information, information from previous programs, etc.), can be used in determining the barrier(s). Sensor information, for instance, can be used to determine and/or support the determination of one or more barriers, such as logistics. If location and/or mobility information collected from sensors of a mobile device of the participant indicates that the participant is homebound (e.g., leaves his or her home less than a predetermined number of times per week, has an average travel radius of below a predetermined threshold, etc.) and/or lives more than a predetermined distance from a location relevant to the user's health regimen and/or condition (e.g., pharmacy, grocery store, etc.), then a logistics barrier may be selected. Information associated with coach-participant communications can additionally or alternatively be analyzed (e.g., manually by the coach, automatically with one or more linguistics algorithms and/or deep learning models, etc.) to assess one or more barriers associated with the participant.

Any or all of the barriers can additionally or alternatively be determined with any or all of: one or more trained models (e.g., as described above), a lookup table, a database, a decision tree, and/or otherwise determined.

Further additionally or alternatively, barriers can be identified in any other suitable ways.

S230 can optionally include communicating the determined barrier(s) to any or all of the coaches and/or other care team members associated with the participant. In some variations, for instance, any or all of: a notification (e.g., text message, email, call, etc.) to any suitable endpoint (e.g., a user device of a coach and/or care team member, a workstation of a care team member, a database such as a medical record and/or clinician note database, a dashboard of a coach and/or care team member, etc.); an updated participant profile (e.g., as viewable at a coach dashboard, at a dashboard accessible by a care team member, etc.); through a communication (e.g., triggered communication, initiated by the coach, requested by a physician, etc.) between coaches and/or care team members; and/or in any other suitable way(s). In preferred variations, the barrier is not communicated to the participant, but alternatively can be.

S230 can optionally include assigning the participant to a cohort of participants (e.g., among all participants in the program, among all participants associated with a coach, etc.), wherein the cohort assignment can be used in any or all of: the selection of one or models, algorithms, rules, or lookup tables (e.g., to select a set of materials to send to the participant, to inform coach communications, etc.); and/or can be used in any other suitable way(s).

In one variation, S230 includes processing the results of a set of one or more surveys administered in S210 (e.g., at a remote computing system) to determine a barrier associated with the participant, wherein the barrier is communicated to at least a coach associated with the participant (e.g., at the participant's profile accessible by the coach) and is optionally used to assign the participant to a cohort of participants based on at least the barrier and optionally the participant condition and/or any other suitable information.

In a second variation, S230 additionally or alternatively includes processing a set of sensor inputs (e.g., location information, motion information, measurements from supplementary devices, etc.) to partially and/or fully determine one or more barriers.

In a specific example, for instance, location information associated with the user (e.g., and collected at the user device) is used to determine whether or not the user is homebound, wherein if it is determined that a participant (equivalently referred to herein as a user) is homebound (e.g., has a low variability in location, has a travel radius below a predetermined threshold, travels to a low number of non-home locations, etc.), a logistics barrier or similar is assigned to the user.

In a third variation, location information associated with the user (e.g., from a location sensor, from demographic information including the user's home address, etc.) is used to determine a social determinant score for the user (e.g., determination that user lives in a food desert based on zip code, etc.), wherein the social determinant can be used to determine one or more barriers (e.g., logistics barrier, cost barrier, etc.).

Additionally or alternatively, S230 can include any other processes and/or be otherwise suitably performed.

4.4 Method: Determining and/or Triggering an Action Based on the Participant Condition and/or the Barrier S240

The method 200 can optionally include determining and/or triggering an action based on the set of inputs S240, wherein the action functions to assist a participant in self-managing his or her participant condition. Additionally, S240 can function to help a participant in achieving his or her health goals, reducing a severity of his or her condition, and/or can perform any other suitable function(s).

S240 is preferably performed in response to S230, but can additionally or alternatively be performed prior to S230, independently and/or in absence of S230, in response to other processes of the method 200, as part of any other processes of the method 200, in parallel with any other processes of the method 200, multiple times during the method 200 (e.g., continuously, at a predetermined frequency, at random intervals, etc.), in response to a trigger, and/or at any other suitable times.

The action can include a set of outputs (e.g., messages, content at the platform, etc.) that the participant receives from his or her coach(es), but can additionally or alternatively be received from other care team members, and/or any other individuals or entities. Further additionally or alternatively, the outputs can be received by coaches (e.g., from care team members), by care team members (e.g., from coaches), and/or can be otherwise determined.

The outputs can include one or more communications, such as communications sent to a participant from a coach (e.g., human coach, non-human coach such as a chat bot, etc.). The communications can include any or all of: messages in a message board (e.g., sent by the coach at a coach dashboard), communications to a user device associated with the participant (e.g., text message, phone call, email, etc.), content (e.g., digital course material, modules, videos, games, etc.), a survey, and/or any other suitable communications.

Additionally or alternatively, the outputs can include one or more materials (e.g., digital materials) sent to a participant as part of the health regimen, such as informational materials (e.g., advising the participant on the medications he is taking, sharing tips associated with the participant's condition and/or barrier, providing exercise tips, providing recipes, etc.); instructions to help a participant obtain his or her medication (e.g., providing a map with directions to the nearest pharmacy); instructions to help a participant use his or her devices (e.g., smart scale, insulin pen, etc.); materials advising the participant on a particular medication based on the participant's insurance policy and/or benefits package; and/or any other suitable materials.

The actions can be any or all of: automatically initiated (e.g., by a remote computing system), partially automatically initiated (e.g., automatically triggering a coach to initiated), manually initiated (e.g., by a coach), and/or any combination. The actions and/or triggers for actions can include any or all of: scheduling (e.g., automatically scheduling) appointments for the participant; refilling (e.g., automatically refilling) prescriptions (and optionally checking in that participant picked up prescription); arranging delivery of prescriptions; facilitating a ride for the user to the pharmacy (e.g., through a ride share service for a homebound participant); administering a copay card to a participant; recommending a client application configured to apply pharmacy discounts; administering a copay card to the participant; sending automatic reminders to a participant at a client application; reassigning the participant to another coach and/or care team member; and/or any other suitable actions.

In some variations, for instance, the set of actions includes automatically determined reminders and/or tools sent to coaches and/or participants, such as any or all of: reminders for coaches to interact with participants based on the participant barrier (e.g., to check in, to intervene, to monitor for certain parameters, etc.); reminders for coaches to provide digital materials to the participant (e.g., based on current medical condition status and known barriers); and/or any other suitable inputs.

In some variations, the action is specifically configured to prevent the need to send reminders (e.g., any reminders, more than a predetermined number of reminders, more than 1 reminder, less than a predetermined frequency of reminders, etc.) to the participant, such as a set of message reminders for the participant to fulfill any or all features (e.g., medication compliance) of the participant's health regimen. In specific examples, for instance, the method is implemented in absence of sending reminders to a participant. This can function, for instance, to overcome limitations of reminder-based approaches (e.g., solely reminder-based approaches), which can result in low participant compliance, higher likelihood of participant quitting the program, and/or other outcomes. Alternatively, the method 200 can implement (e.g., trigger) any number of actions.

Any or all of the actions can be automatically determined, partially automatically determined (e.g., autofilling a communication for the coach), manually determined (e.g., by a coach), and/or any combination of the above.

Additionally or alternatively, any or all of the actions can be triggered based on features of the participant, such as particular barriers associated with the participant. In some examples, for instance, an action involving automatically prompting (e.g., through a notification, through pulling up a 3^{rd} party rideshare application, through initiating the 3^{rd} party rideshare application, etc.) the user to engage with a rideshare application in order to travel to a pharmacy to pick up his or her prescription can be specifically triggered for a user associated with a logistics barrier. In other examples, for instance, an action involving automatically controlling a vehicle to transport the user to a destination (e.g., automatically selected to be the nearest pharmacy location, grocery store, etc.) can be specifically triggered for a user associated with a logistics barrier. In variants, sensors in the vehicle can monitor the user during transport, wherein the measurements can be used to determine the user state, future responses, future interventions, and/or otherwise used. Additionally or alternatively, post-transportation user data for the user can be determined and used to determine user state, future responses, future user interventions, retrain the model (e.g., the one that indicated that transportation should be an intervention), and/or otherwise used. In other examples, for instance, an action involving prompting the sending of a copay card and/or prompting the participant to enroll in a pharmacy/prescription savings program can be triggered for a participant associated with a cost barrier. Additionally or alternatively, actions can be otherwise determined.

Additionally or alternatively, any or all of the actions can be determined based on a set of one or more models, such as a models selected based on one or both the participant condition and the barrier. In an example, each model in a set of models is associated with a set of barriers (e.g., a logistics barrier, a range of values for a logistics barrier, multiple barriers, etc.).

In a first set of variations, S240 is performed with a model trained to determine (e.g., predict) a most impactful action for a particular user and/or users similar to the user (e.g., in barrier(s), in goal(s), in background, etc.), wherein the most impactful action is predicted to lead to the greatest progress and/or outcome (e.g., in advancing the participant toward his or her goal) for the participant in his or her health regimen. The model can include a multiclass classifier (e.g., where each class can be different outcomes, outputs, actions, responses, etc.).

In a set of specific examples, S240 includes calculating an impact score for a set of potential actions (e.g., with uplift modeling), wherein the action triggered corresponds to the highest impact score.

In a second set of variations, where medication barriers are detected to exist, the coaches can be advised to follow an MTM care process to apply motivational interviewing techniques to understand the participant's medication needs, develop strategies to overcome barriers, and follow up to ensure the participant achieves their therapy goals. When multiple medication barriers exist, coaches can optionally work with the participants to create an action list that prioritizes the problems based on their participant's preference and clinical needs.

In a third set of variations, a set of barriers are used to determine one or more actions for the participant (e.g., based on a set of models, based on a lookup table, based on a decision tree, etc.).

S240 can additionally or alternatively include any other processes and/or be otherwise suitably performed.

### 4.5 Method: Updating a Set of Models Based on a Set of Outputs S250

The method 200 can optionally include updating (e.g., retraining) on any or all of the models described previously based on a set of outputs S250, which functions to enable the system 100 to learn from the complex relationships involved in adherence to a health regimen with various different participant conditions.

S250 is preferably performed in response to S240, but can additionally or alternatively be performed prior to S210 (e.g., in initially training one or more models), prior to S240, independently and/or in absence of S240, in response to other processes of the method 200, as part of any other processes of the method 200, in parallel with any other processes of the method 200, multiple times during the method 200 (e.g., continuously, at a predetermined frequency, at random intervals, etc.), in response to a trigger, and/or at any other suitable times.

S250 preferably includes determining a success (e.g., through a success metric, through a progress metric, through a goal achievement metric, etc.) of the participant in achieving his or her set of health goals, wherein the results of this can be used to further train one or more deep learning models. Success in achieving health goals is preferably informed based on participant information continuously and/or repeatedly connected through S210, such as any or all of: evidence of medication adherence or non-adherence; sensor measurements indicating the current status of the participant condition (e.g., hemoglobin levels); clinician feedback (e.g., status at latest appointment); coach feedback (e.g., participant participation level in the program); and/or any other suitable information.

In some variations, for instance, any or all of a set of models implemented in the method 200 can be continuously (e.g., with new participants, at a predetermined set of time intervals, etc.) updated with data from participants.

Additionally or alternatively, S250 can include any other suitable processes and/or be otherwise suitably performed.

### 4.6 Method: Variations

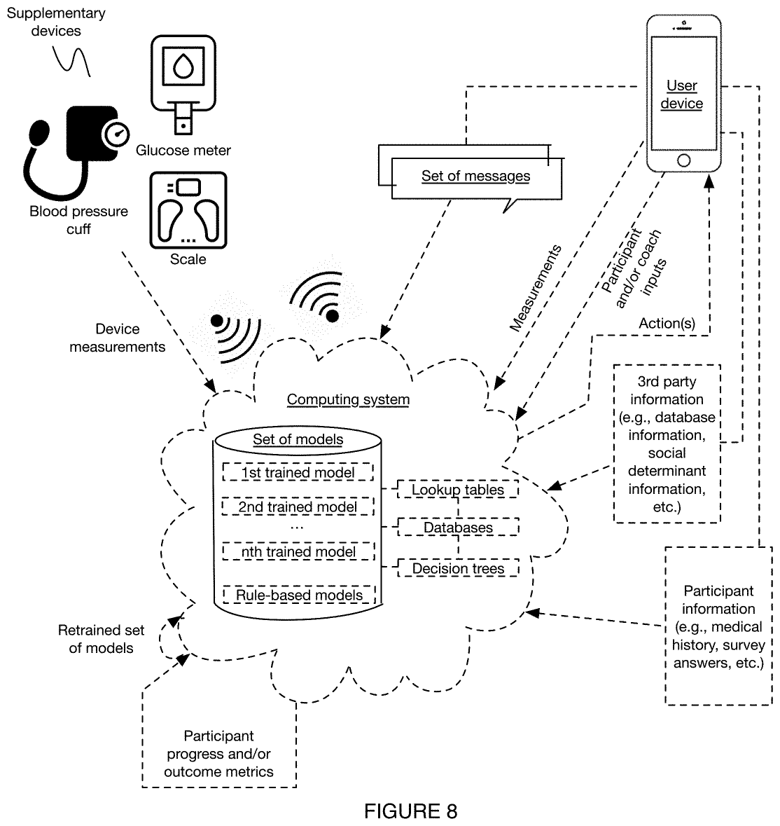

In a set of variations of the method 200 (e.g., as shown in FIG. 8), the method includes: collecting participant information S210, wherein the participant information includes any or all of: baseline information (e.g., demographic information, medical information such as medical record information and/or clinician notes, etc.), participant information used to assess one or more barriers (e.g., collected after the baseline information through a set of surveys), and optionally sensor information (e.g., from a set of devices associated with the participant) and/or adherence information (e.g., evidence/record of participant taking medication); determining a participant condition (e.g., medical condition) based on the participant information and optionally additional information from any or all of: a physician or care team member associated with the participant, a database (e.g., EHR database), and/or any other suitable information; determining a barrier associated with the participant based at least in part on participant information collected in S210, such as the results of participant surveys; and determining a set of actions for engaging with the participant S230, wherein the set of inputs are provided by and/or recommended to the coaches (e.g., resulting from a set of deep learning models) to send to the participants to help them best manage their condition(s) in light of their barrier(s). Additionally, the method 200 can include any or all of: repeating S210 throughout the duration of the participant's participation in the program; updating a set of models based on a set of outputs S250, such as participant progress in achieving a health goal; and/or any other suitable processes.

In a first set of specific examples wherein a cost barrier is identified, coaches can provide inputs to participants and/or coaches and care team members can share resources on medication specific cost reduction strategies, such as any or all of: manufacturer's copay cards, prescription drug assistance programs, generic alternatives, and retail prescription savings programs (e.g., Walmart's $4 generic list).

In a second set of specific examples, wherein a forgetfulness barrier is identified, coaches can optionally provide reminders to participants, but can additionally or alternatively look further into potential other barriers, such as the health beliefs, medication regimen complexity, and external barriers that contribute to medication-taking behavior.

In a third set of specific examples, wherein a logistical barrier is identified (e.g., participant experiences logistical challenges to obtaining his medications from a community pharmacy due to busy schedules, a lack of transportation, or disabilities), any or all of the following inputs can be initiated: pharmacist-led interventions to synchronize the refill dates for multiple prescriptions, mail order pharmacy, switching from 30 to 90-day refills, and adherence packaging solutions.

In a fourth set of specific examples, wherein a side effects barrier is identified, a communication can be initiated (e.g., between a coach and a participant, between a participant and a care team member, between a coach and a care team member, between two participants such as a two participants in a same condition and/or barrier cohort, etc.), wherein the communication can include any or all of: participants sharing their medication experiences, a provider optimizing medication therapy by identifying a therapeutic alternative, provider prescribing an extended release formulation, a coach or care team member providing medication-taking strategies to improve the tolerability of drug therapy, and/or any other suitable communications or actions.

In a second set of variations, additional or alternative to the first, any or all of the method 200 is performed with a set of trained models, such as a set of trained machine learning models.

In a set of specific examples, a first trained model can be used to determine and/or trigger any or all of the set of specific examples. Additionally or alternatively, the first trained model and/or any other trained models can be used for any or all of: determining a barrier associated with the participant, determining a goal of the participant, assigning the participant to a coach, and/or any other actions.

Additionally or alternatively, the method 200 can include any other suitable processes or embodiments.

Different processes and/or elements discussed above can be performed and controlled by the same or different entities. In the latter variants, different subsystems can communicate via: APIs (e.g., using API requests and responses, API keys, etc.), requests, and/or other communication channels.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), concurrently (e.g., in parallel), or in any other suitable order by and/or using one or more instances of the systems, elements, and/or entities described herein.

Although omitted for conciseness, the preferred embodiments include every combination and permutation of the various system components and the various method processes, wherein the method processes can be performed in any suitable order, sequentially or concurrently.

As a person skilled in the art will recognize from the previous detailed description and from the figures and claims, modifications and changes can be made to the preferred embodiments of the invention without departing from the scope of this invention defined in the following claims.

We claim:

1. A method comprising:
calculating constraints for each user in a set of users based on a set of user data, wherein the set of user data comprises:
user inputs collected using a user device;
geographic location measurements associated with the user, wherein the geographic location measurements are collected with a first set of sensors mounted to the user device; and
biometric parameters associated with a health condition of the user measured with a second set of sensors mounted to a supplementary device;
training each of a set of predictive models, each predictive model associated with a set of constraints, comprising:
selecting a subset of users associated with the respective set of constraints from the set of users; and
training the predictive model on user data for the subset of users; and
for a given user,
selecting a predictive model from the set, the selected predictive model sharing constraints with the given user;
using the selected predictive model, determining a user output based on the set of user data; and
presenting the user output on the user device.

2. The method of claim 1, further comprising, for the given user:
measuring a user response; and
updating the predictive model with feedback based on the user response.

3. The method of claim 1, wherein the set of constraints associated with each predictive model comprises a set of constraint ranges, and wherein selecting a subset of users associated with the respective set of constraints from the set of users comprises selecting users associated with constraint values falling within the set of constraint ranges.

4. The method of claim 1, wherein each of the set of predictive models comprises a multiclass classifier, wherein each class comprises a different candidate output, wherein the selected predictive model is trained to predict an impact of each candidate output on the user, wherein the candidate output with a highest predicted impact is used as the user output.

5. The method of claim 1, wherein training each of the set of predictive models comprises implementing uplift modeling to select the user output with a highest predicted impact.

6. The method of claim 5, wherein training each of the set of predictive models is based on a mapping between each user output and user data for the subset of users.

7. The method of claim 1, wherein calculating constraints for each user in a set of users comprises calculating a logistics constraint when the geographic location measurements for the user exhibit low variability.

8. The method of claim 7, wherein calculating the logistics constraint comprises calculating a travel radius parameter based on the geographic location measurements and determining that the travel radius parameter is below a predetermined threshold.

9. The method of claim 1, wherein the determined user output is associated with travel, the method further comprising:
automatically controlling a vehicle to transport the given user to an automatically selected destination.

10: The method of claim 9, wherein the destination is a location of a pharmacy proximal to the user.

11. The method of claim 1, further comprising, for a given user:
analyzing the user data to determine whether the user is exhibiting aberrant behavioral patterns as compared to a user population that the user belongs to; and
automatically operating the user device when the analysis indicates that the user is exhibiting an aberrant behavior pattern.

12. The method of claim 11, wherein automatically operating the user device comprises automatically operating the user device to sample different parameters when the analysis indicates that the user is exhibiting an aberrant behavior pattern.

13. The method of claim 1, wherein, for each user in the set of users, the constraints are further calculated based on a set of communication parameters determined based on a set of messages exchanged between the user and a coach assigned to the user.

14. The method of claim 13, wherein the set of communication parameters comprises at least one of: a content associated with the set of messages, a length of the messages, and a frequency of the messages.

15. A system comprising:
a user device for:
collecting a set of user inputs associated with a user;
transmitting the set of user inputs to a remote computing system; and
displaying a user interface output received from the remote computing system;
a first set of sensors for collecting geographic location measurements associated with the user, wherein the first set of sensors are mounted to the user device;
a second set of sensors for measuring biometric parameters associated with a health condition of the user, wherein the second set of sensors are mounted to a secondary device; and
the remote computing system, comprising a memory and a processing system coupled to the memory programmed with executable instructions for:
receiving user data, wherein the user data comprises the set of user inputs, the geographic location measurements, and the biometric parameters;
calculating constraints for the user based on at least one of: the set of user inputs, the geographic location measurements, or the biometric parameters;
selecting a predictive model from a set of predictive models based on the constraints for the user, wherein each predictive model is associated with a set of constraints, and wherein the selected predictive model is trained on historic user data associated with a subset of users sharing the set of constraints;
using the selected predictive model, determining a user interface output; and
controlling the user device based on the selected user interface output.

16. The system of claim 15, wherein the executable instructions for determining a user interface output comprises executable instructions for:
using the selected predictive model to predict an impact of each of a set of candidate user outputs on the user; and
selecting a user interface output with the highest predicted impact of the set of candidate user outputs.

17. The system of claim 16, wherein the selected predictive model implements uplift modeling to predict the impact of each of the set of candidate user outputs.

18. The system of claim 16, wherein the set of candidate user outputs comprises a set of messages.

19. The system of claim 15, further comprising executable instructions for:
analyzing user data for the user to determine whether the individual user is exhibiting aberrant behavioral patterns as compared to the subset of users; and
automatically operating the user device when the analysis indicates that the user is exhibiting an aberrant behavior pattern.

20. The system of claim 19, wherein automatically operating the user device comprises automatically adjusting the determined user interface output when the analysis indicates that the user is exhibiting an aberrant behavior pattern.
